(19) United States
(12) Patent Application Publication
(10) Pub. No.: US 2015/0123673 A1
(43) Pub. Date: May 7, 2015

(54) BLOOD TESTING METER WITH A FOOL-PROOFING FUNCTION

(71) Applicant: Chun-Tung (Jack) CHEN, Taipei City (TW)

(72) Inventor: Chun-Tung (Jack) CHEN, Taipei City (TW)

(21) Appl. No.: 14/528,169

(22) Filed: Oct. 30, 2014

(30) Foreign Application Priority Data
Nov. 6, 2013 (TW) 102220685

(51) Int. Cl. G01R 31/04; A61B 5/145

(52) U.S. Cl. CPC G01R 31/04; A61B 5/14532
USPC 324/538

(57) ABSTRACT

A blood testing meter with a fool-proofing function has a measuring display unit, a connector and a blocking structure. The connector is mounted in the measuring display unit and has at least one guiding groove, and the at least one guiding groove is formed through an outer side of the measuring display unit. The blocking structure is mounted on the outer side of the measuring display unit and is adjacent to an opening of the connecting groove. When the blood test strip is inserted from a wrong direction into the connector, the slot would be engaged with the blocking structure to prevent the blood test strip from being inserted into the connector.

### BACKGROUND OF THE INVENTION

1. Field of the Invention

The prevention relates to a blood testing meter, especially a blood testing meter with a fool-proofing function that can prevent the test strip from being erroneously inserted into the blood testing meter.

2. Description of the Prior Arts

A conventional blood glucose meter is used with a conventional blood test strip. In use, blood of a user is dripped on the conventional blood test strip, and the glucose of the blood is reacted with a sensing layer of the conventional blood test strip. The reaction signal is transmitted to the conventional blood glucose meter. The reaction signal is analyzed by the conventional blood glucose meter and is transferred to a corresponding value displayed on a screen of the conventional blood glucose meter to enable the user to obtain the glucose value.

The conventional blood test strip and the conventional blood glucose meter have a specific connecting direction. When the conventional blood test strip is inserted from a wrong direction into the conventional blood glucose meter, the conventional blood test strip doesn't connect electrically to the conventional blood glucose meter, and the conventional blood glucose meter is invalid in testing. However, both the conventional blood glucose meter and the blood test strip don't have any equipment to prevent erroneous insertions, so the conventional blood glucose meter can't receive and transfer the blood testing signal when the conventional blood test strip is inserted from the wrong direction.

To overcome the shortcomings, the present invention provides a blood testing meter with a fool-proofing function to mitigate or obviate the aforementioned problems.

### SUMMARY OF THE INVENTION

The main objective of the present invention is to provide a blood testing meter with a fool-proofing function that can prevent the test strip from inserting erroneously into the blood testing meter.

A blood testing meter with a fool-proofing function comprises a measuring display unit, a connector and a blocking structure. The connector is mounted in the measuring and has at least one guiding groove, and the at least one guiding groove is formed through an outer side of the measuring display unit. The blocking structure is mounted on the outer side of the measuring display unit and is adjacent to an opening of the connector.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a first embodiment of a blood testing meter with a fool-proofing function in accordance with the present invention;

FIG. 2 is a right operational top view of the blood testing meter in FIG. 1;

FIG. 3 is a wrong operational top view of the blood testing meter in FIG. 1;

FIG. 4 is a perspective view of a second embodiment of a blood testing meter with a fool-proofing function in accordance with the present invention;

FIG. 5 is a right operational top view of the blood testing meter in FIG. 4; and

FIG. 6 is a wrong operational top view of the blood testing meter in FIG. 4.

### DETAILED DESCRIPTION OF THE PREFERRED EMBODIMENTS

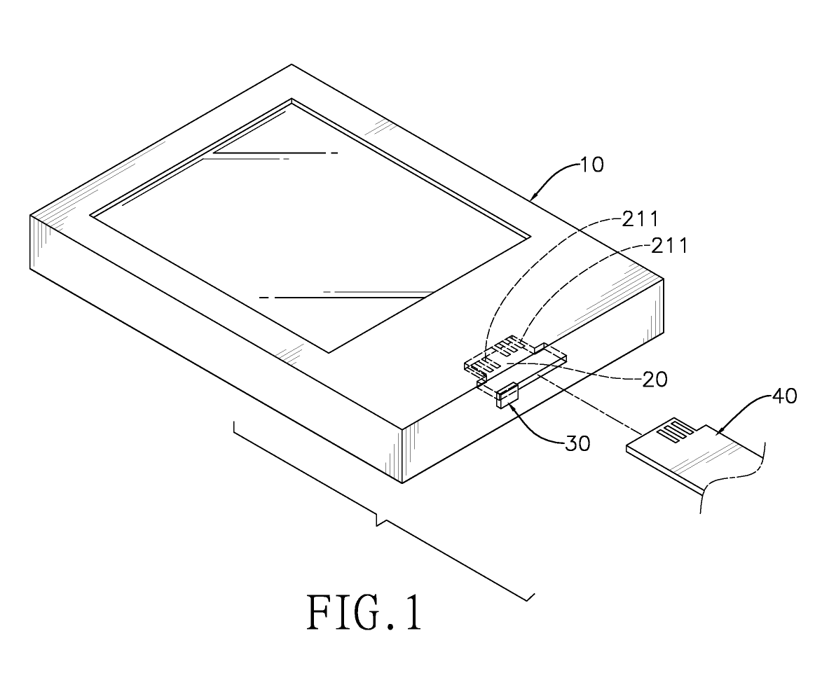

With reference to FIG. 1, a first embodiment of a blood testing meter with a fool-proofing function in accordance with the present invention has a measuring display unit 10, a connector 20 and a blocking structure 30.

With reference to FIG. 1, the measuring display unit 10 is used to receive and analyze electrical signals, and transfer corresponding values that have been calculated from the electrical signals and display the corresponding values on a screen of the measuring display unit 10.

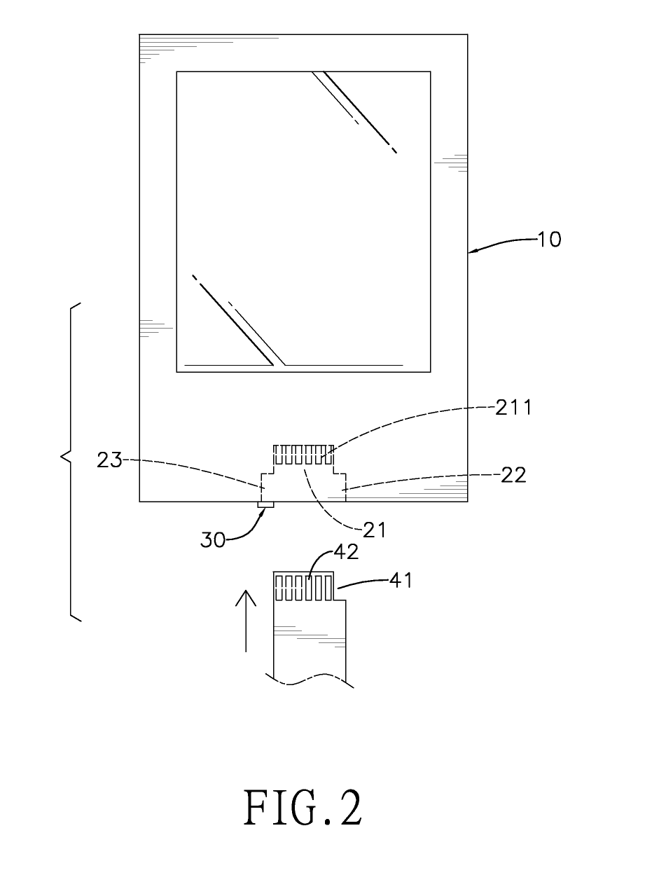

With reference to FIGS. 1 and 2, the connector 20 is mounted in the measuring display unit 10 and has a connecting groove 21, a first guiding groove 22 and a second guiding groove 23. The connecting groove 21 is formed through an outer side of the measuring display unit 10 and has an internal surface and multiple metal pins 211 mounted on the internal surface of the connecting groove 21 at intervals. The first guiding groove 22 and the second guiding groove 23 are formed through the outer side of the measuring display unit 10 beside the connecting groove 21, communicate with the connecting groove 21, and are parallel with each other.

The blocking structure 30 may be made of plastic, is mounted on the outer side of the measuring display unit 10 adjacent to an opening of the connecting groove 21 of the connector 20 to cover the second guiding groove 23.

In use, with reference to FIG. 2, a blood test strip 40 has a slot 41 and multiple electrodes 42. The slot 41 is formed through a front side of the blood test strip 40. The electrodes 42 are respectively mounted on a top surface and a bottom surface of the blood test strip 40 at the front side of the blood test strip 40. The front side of the blood test strip 40 is inserted into the connector 20 by a user via the opening of the connecting groove 21. The slot 41 of the blood test strip 40 engages with the first guiding groove 22 of the connector 20. The electrodes 42 that are mounted on the front side of the blood test strip 40 are mounted in the connecting groove 21 of the connector 20, and are respectively and electrically connected to the metal pins 211 of the connecting groove 21.

With reference to FIGS. 1 and 2, the metal pins 211 of the connecting groove 21 are formed on an internal surface of the connecting groove 21. Three of the metal pins 211 are mounted on a top segment of the internal surface of the connecting groove 21, the other metal pins 211 are mounted on a bottom segment of the internal surface of the connecting groove 21. The electrodes 42 of the blood test strip 40 correspond to the metal pins 211 of the connecting groove 21, so the electrodes 42 of the blood test strip 40 have to be inserted into the connector 20 from a right direction for electrically connecting to the metal pins 211.

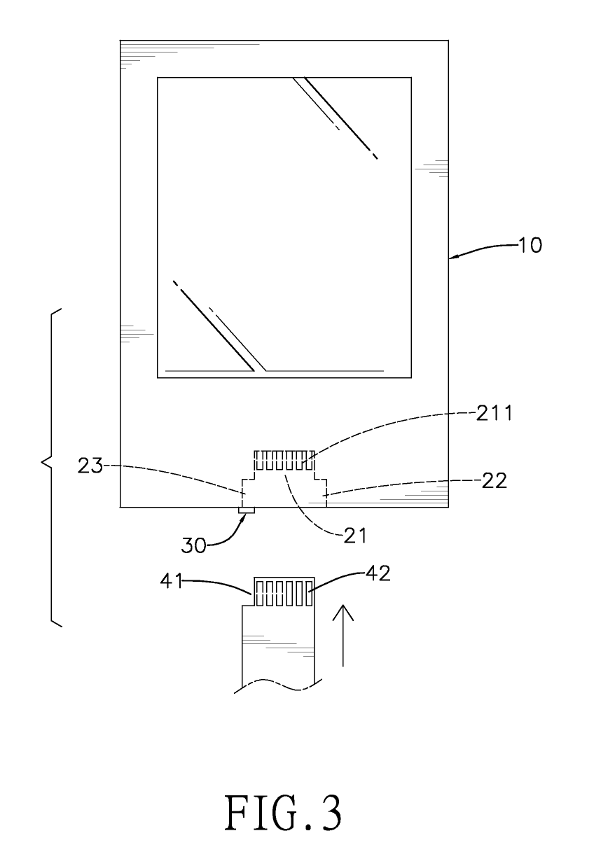

With reference to FIG. 3, when the blood test strip 40 is inserted into the connector 20 from a wrong direction, the slot 41 aligns with the second guiding groove 23. Then, the slot 41 will be engaged with the blocking structure 30 to prevent the blood test strip 40 from being inserted into the connector 20.

The connector 20 and the blocking structure 30 are mounted on the measuring display unit 10 and are connected to the blood test strip 40. In assembly, the blood test strip 40 can only be inserted into the connector 20 when the slot 41 aligns with the first guiding groove 22, otherwise the blood test strip 40 would be obstructed by the blocking structure 30. So the present invention has a function of preventing the blood test strip from being erroneously inserted into the blood testing meter.

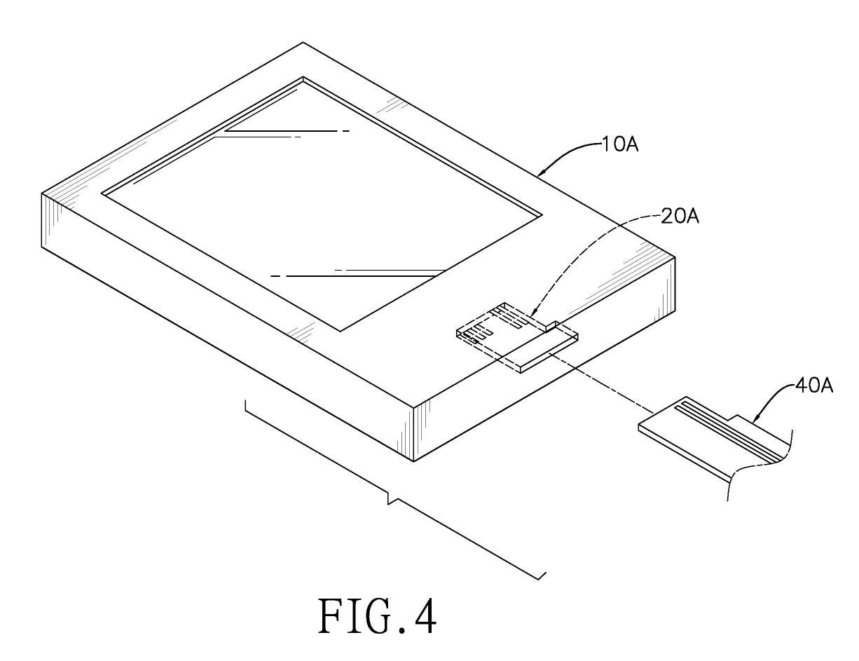

With reference to FIG. 4, a second embodiment of a blood testing meter with a fool-proofing function in accordance with the present invention has a measuring display unit 10A and a connector 20A.

The connector 20A is mounted in the measuring display unit 10A and has a connecting groove 21A and a guiding groove 22A. The connecting groove 21A is formed through an outer side of the measuring display unit 10 and has an internal surface and multiple metal pins 211A mounted on the internal surface of the connecting groove 21A. The guiding groove 22A is formed through the outer side of the measuring display unit 10A and communicates with the connecting groove 21A.

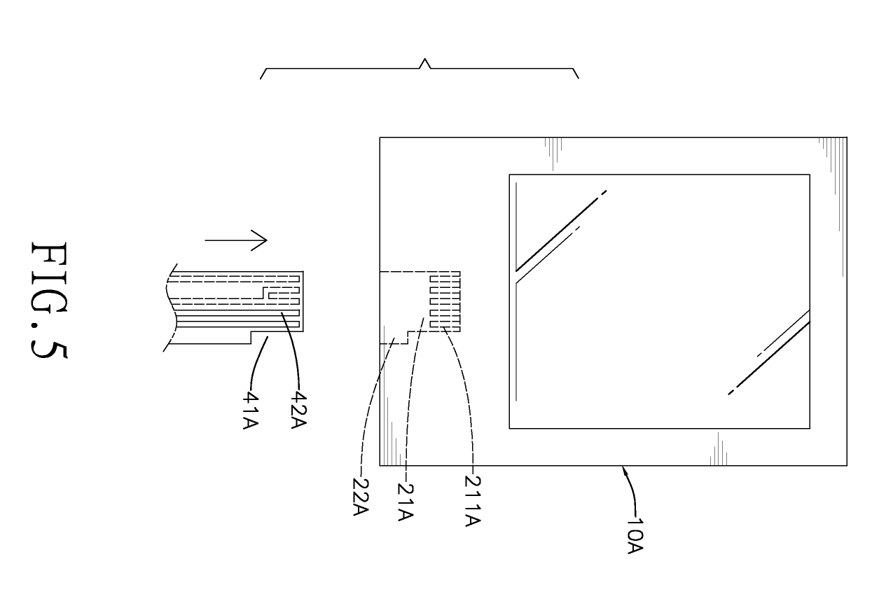

With reference to FIG. 5, a blood test strip 40A has a slot 41A and multiple electrodes 42A. The slot 41A is formed through a front side of the blood test strip 40A. The electrodes 42A are respectively mounted on a top surface and a bottom surface of the blood test strip 40A at the front side of the blood test strip 40A. In use, the blood test strip 40A is inserted into the connector 20A by a user. The slot 41A of the blood test strip 40A engages with the guiding groove 22A of the connector 20A. The electrodes 42A that are mounted on the front side of the blood test strip 40A are mounted in the connecting groove 21A of the connector 20A, and are respectively and electrically connected to the metal pins 211A.

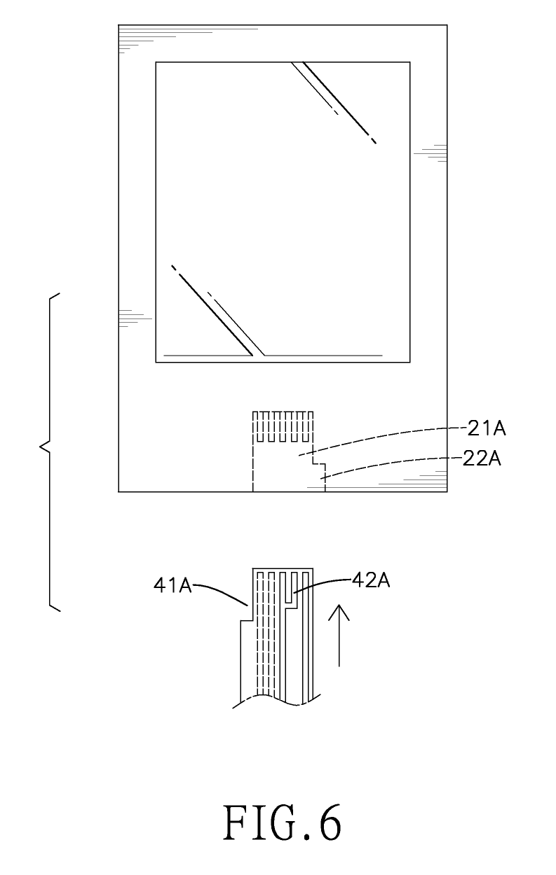

With reference to FIG. 6, when the blood test strip 40A is inserted into the connector 20A from a wrong direction, the slot 41A would be engaged with the outer side of the measuring display unit 10A to prevent the blood test strip 40A from being inserted into the connector 20A.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A blood testing meter with a fool-proofing function comprising:
a measuring display unit;
a connector mounted in the measuring display unit and having
at least one guiding groove formed through an outer side of the measuring display unit; and
a blocking structure mounted on the outer side of the measuring display unit and being adjacent to an opening of the connector.

2. The blood testing meter as claimed in claim 1, wherein the connector has a first guiding groove and a second guiding groove, the first guiding groove and the second guiding groove are formed through the outer side of the measuring display unit and are parallel with each other.

3. The blood testing meter as claimed in claim 1, wherein the blocking structure is made of plastic and covers the second guiding groove.

4. The blood testing meter as claimed in claim 2, wherein the blocking structure is made of plastic and covers the second guiding groove.

5. The blood testing meter as claimed in claim 1, wherein
the connector has
a connecting groove formed on an inner surface of the measuring display unit and having
an internal surface; and
multiple metal pins mounted on the internal surface of the connecting groove at intervals.

6. The blood testing meter as claimed in claim 3, wherein
the connector has
a connecting groove formed on an inner surface of the measuring display unit and having
an internal surface; and
multiple metal pins mounted on the internal surface of the connecting groove at intervals.

7. The blood testing meter as claimed in claim 4, wherein
the connector has
a connecting groove formed on an inner surface of the measuring display unit and having
an internal surface; and
multiple metal pins mounted on the internal surface of the connecting groove at intervals.

8. The blood testing meter as claimed in claim 1, wherein the connector has a guiding groove formed through the outer side of the measuring display unit and communicates with the connecting groove.
